(19) United States
(12) Patent Application Publication
(10) Pub. No.: US 2022/0008585 A1
(43) Pub. Date: Jan. 13, 2022

(54) COVID-19 DISINFECTANT HAND-HELD PORTABLE MISTING SPRAYER SYSTEM METHOD AND DEVICES

(71) Applicants: Steven B. Yamashiro, Rancho Dominguez, CA (US); Enrique Prenda Alabata, Rancho Dominguez, CA (US)

(72) Inventors: Steven B. Yamashiro, Rancho Dominguez, CA (US); Enrique Prenda Alabata, Rancho Dominguez, CA (US)

(21) Appl. No.: 17/016,348

(22) Filed: Sep. 9, 2020

Related U.S. Application Data
(63) Continuation-in-part of application No. 16/923,037, filed on Jul. 7, 2020, now abandoned.

(51) Int. Cl. A61L 2/22; A61L 2/24; A61L 2/26

(52) U.S. Cl. CPC A61L 2/22; A61L 2101/20; A61L 2/26; A61L 2/24

(57) ABSTRACT

The embodiments disclose a method including providing a hand-held portable misting sprayer for applying HOCL to surfaces of objects for destroying airborne and surface contacted COVID-19 SARS-CoV-2 virus and other viruses, coupling a portable rechargeable battery to the hand-held portable misting sprayer for supplying power to operate elements of the hand-held portable misting sprayer, coupling a reservoir to the hand-held portable misting sprayer for containing a volume of HOCL for processing in spraying operations, coupling a motor to the hand-held portable misting sprayer for pressurizing HOCL through a spray nozzle for creating a mist, coupling electronic processing and communication modules to the hand-held portable misting sprayer and components for gathering, recording, processing data gathered, and transmitting the data to external digital devices wirelessly, and providing a hand-held/portable misting sprayer app for use on user digital devices for receiving and processing data transmitted from the hand-held portable misting sprayer.

### CROSS-REFERENCE TO RELATED APPLICATIONS

This continuation-in-part application is based on pending U.S. Non-Provisional application Ser. No. 16/923,037 filed Jul. 7, 2020, entitled “COVID-19 DISINFECTANT MISTING SYSTEM METHOD AND DEVICES”, by Steven B. Yamashiro.

### BACKGROUND

Recent experience during the COVID-19 pandemic has concentrated on personal protective equipment. The restrictions shutting down businesses and facilities for large gatherings pointed out one shortcoming of the approach to slow the spread of the infections and beat the COVID-19 plague. People have built up anxiety and resentment at the inability to gather for dinners, a night at the movies, attending places of worship, travel and even going to work. Even the recent relaxing of restrictions has placed a manpower burden on business owners to manually disinfect all surfaces. But the virus that causes COVID-19 is air borne, hence the mask and social distancing. Those airborne viruses settle back on the just cleaned surfaces within minutes. What is needed is a thorough disinfecting system of not only surfaces but the air itself. A thorough disinfecting system that is fast and can easily be repeated during the day and night to create a safe environment in large commercial gathering places to truly provide a safe area for social gatherings and work places.

### BRIEF DESCRIPTION OF THE DRAWINGS

FIG. 1 shows a block diagram of an overview flow chart of applying a non-toxic HOCL COVID-19 disinfectant mist for large commercial areas of one embodiment.

FIG. 2 shows a block diagram of an overview flow chart of positioning a HOCL misting system device for dispersing an aerosol mist application of a non-toxic HOCL solution of one embodiment.

FIG. 3 shows for illustrative purposes only an example of a HOCL misting system control app of one embodiment.

FIG. 4 shows for illustrative purposes only an example of a HOCL misting into an airplane ventilation system of one embodiment.

FIG. 5 shows for illustrative purposes only an example of a public restroom HOCL disinfectant mist of one embodiment.

FIG. 6 shows for illustrative purposes only an example of a movie theater HOCL disinfectant mist of one embodiment.

FIG. 7 shows for illustrative purposes only an example of misting diffused HOCL droplets of one embodiment.

FIG. 8 shows for illustrative purposes only an example of HOCL contact with a SARS-CoV2 virus cell lipid layer of one embodiment.

FIG. 9 shows for illustrative purposes only an example of a hand-held/portable misting sprayer of one embodiment.

FIG. 10 shows a block diagram of an overview of a hand-held/portable misting sprayer system of one embodiment.

FIG. 11 shows for illustrative purposes only an example of a hand-held/portable misting sprayer exterior view of one embodiment.

FIG. 12 shows for illustrative purposes only an example of a hand-held/portable misting sprayer interior view of one embodiment.

FIG. 13 shows for illustrative purposes only an example of a hand-held/portable misting sprayer app of one embodiment.

FIG. 14 shows for illustrative purposes only an example of a hand-held/portable misting sprayer network platform of one embodiment.

FIG. 15 shows for illustrative purposes only an example of electronic processing and communication modules of one embodiment.

FIG. 16 shows for illustrative purposes only an example of a hand-held/portable misting sprayer application on a ship's bridge of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview:

It should be noted that the descriptions that follow, for example, in terms of a COVID-19 disinfectant misting system method and devices is described for illustrative purposes and the underlying system can apply to any number and multiple types of large commercial areas. In one embodiment of the present invention, the COVID-19 disinfectant misting system method and devices can be configured using a non-toxic HOCL COVID-19 disinfectant. The COVID-19 disinfectant misting system method and devices can be configured to include direct misting contact and can be configured to include indirect misting applications using air ventilation system dispersing using the present invention.

The term “HOCL” used herein is an abbreviation for Hypochlorous Acid and consistently has that same meaning.

The terms “COVID-19 disinfectant misting system”, “HOCL misting system” and “HOCL mister” are all used interchangeably herein without any change in meaning.

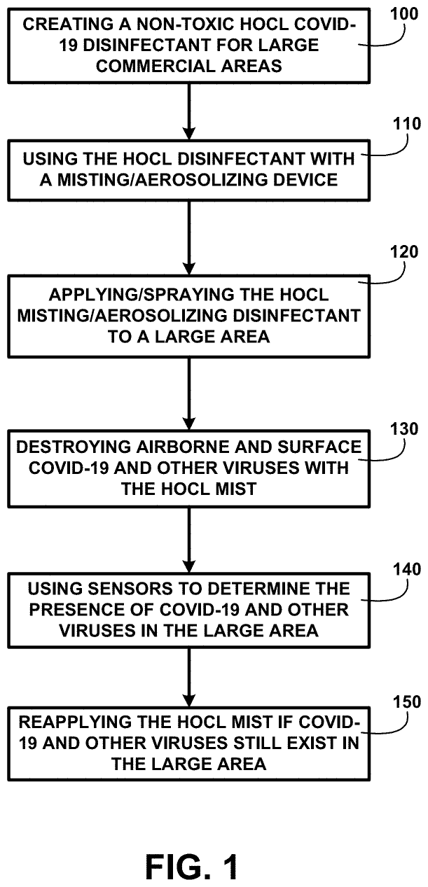

FIG. 1 shows a block diagram of an overview flow chart of applying a non-toxic HOCL COVID-19 disinfectant mist for large commercial areas of one embodiment. FIG. 1 shows creating a non-toxic HOCL COVID-19 disinfectant for large commercial areas 100. Fast and convenient disinfecting of large commercial areas can be repeated during the day and night using the HOCL disinfectant with a misting/aerosolizing device 110.

Both surfaces and the air within the large commercial area are disinfected with applying/spraying the HOCL misting/aerosolizing disinfectant to a large area 130. The HOCL disinfectant is used effectively for destroying airborne and surface COVID-19 and other viruses with the HOCL mist 130. The HOCL disinfectant misting/aerosolizing device includes using sensors to determine the presence of COVID-19 and other viruses in the large area 140. The use of sensors to determine the presence of COVID-19 provides a safety notice for reapplying the HOCL mist if COVID-19 and other viruses still exist in the large area 150 of one embodiment.

### DETAILED DESCRIPTION:

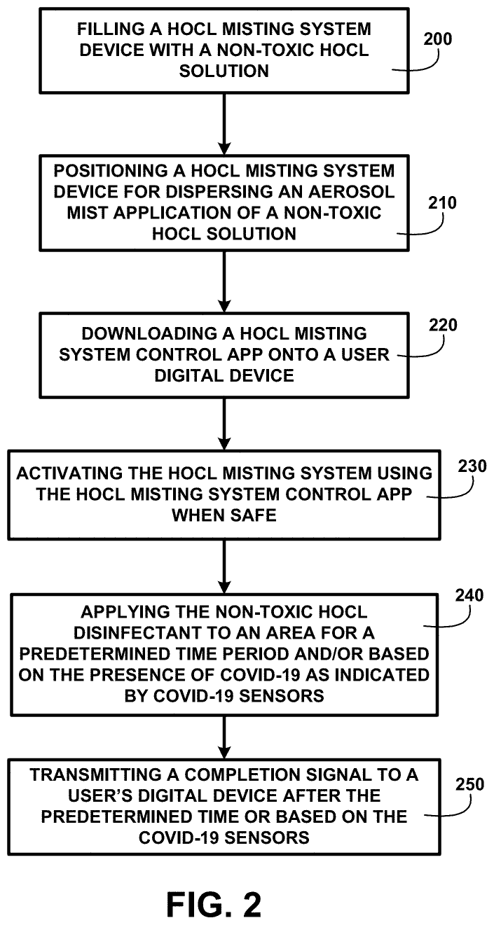

FIG. 2 shows a block diagram of an overview flow chart of positioning a HOCL misting system device for dispersing an aerosol mist application of a non-toxic HOCL solution of one embodiment. FIG. 2 shows a process for filling a HOCL misting system device with a non-toxic HOCL solution 200. Complete disinfecting coverage is achieved with positioning a HOCL misting system device for dispersing an aerosol mist application of a non-toxic HOCL solution 210. A user has access for controlling the HOCL misting system device with downloading a HOCL misting system control app onto a user digital device 220.

The user starts an aerosol mist application when activating the HOCL misting system using the HOCL misting system control app when safe 230 pushing a start button. An aerosol mist application is performed applying the non-toxic HOCL disinfectant to an area for a predetermined time period and/or based on the presence of COVID-19 as indicated by COVID-19 sensors 240. The HOCL misting system control app notifies the user with transmitting a completion signal to a user's digital device after the predetermined time or based on the COVID-19 sensors 250 of one embodiment.

### A HOCL Misting System Control App:

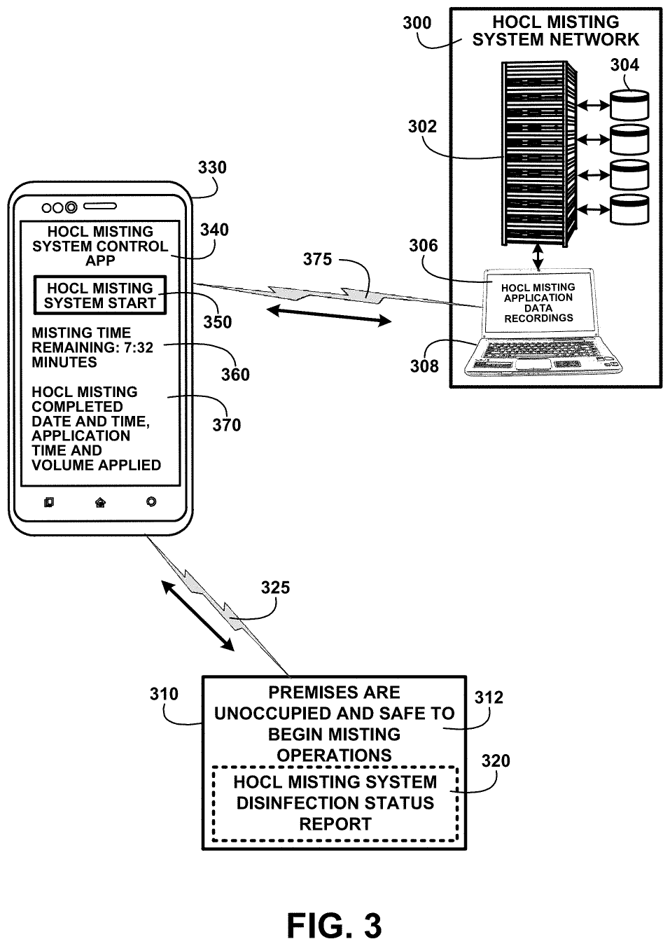

FIG. 3 shows for illustrative purposes only an example of a HOCL misting system control app of one embodiment. FIG. 3 shows a HOCL misting system network 300 used for documenting the disinfection applications. A digital server 302 and a plurality of databases 304 is used for maintain disinfection data with HOCL misting application data recordings 306. A network computer 308 receives collected data from a HOCL misting system control app 340. The collected data includes information from large commercial area occupancy status sensors 310. The large commercial area status includes when premises are unoccupied and safe to begin misting operations 312. A HOCL misting system disinfection status report 320 is transmitted 325 to a user digital device 330 on the HOCL misting system control app 340.

When the HOCL misting system disinfection status report 320 indicates it is safe to begin operations a HOCL misting system start 350 button is activated on the HOCL misting system control app 340. The user pushes the HOCL misting system start 350 button. The HOCL misting system control app 340 includes a display of for example misting time remaining: 7:32 minutes 360. The HOCL misting system control app 340 includes a display of a HOCL misting completed date and time, application time volume applied 370. A HOCL misting system control app transmits a completion signal and transmits a HOCL misting a data recording signal 375 of one embodiment.

A HOCL Misting into an Airplane Ventilation System:

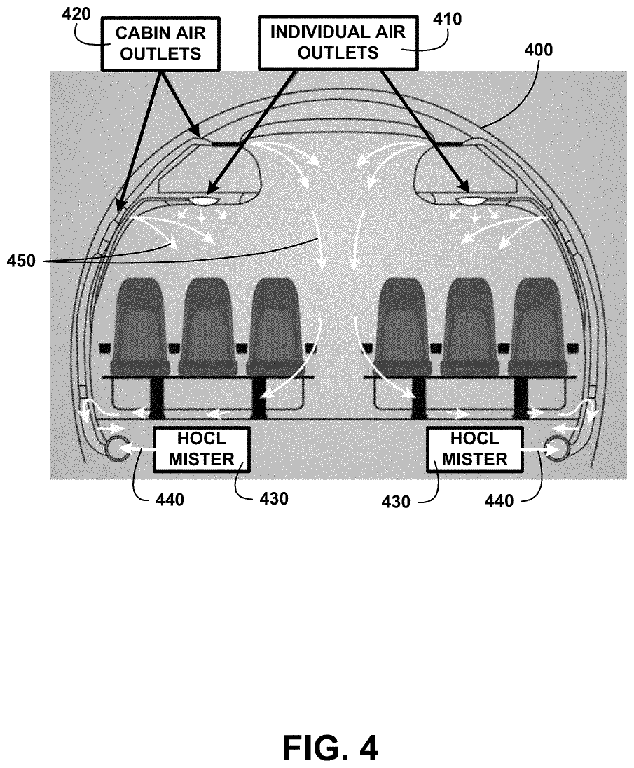

FIG. 4 shows for illustrative purposes only an example of a HOCL misting into an airplane ventilation system of one embodiment. FIG. 4 shows a view of an interior of an airplane 400. Commercial and private planes include a ventilation system to provide a change of air and temperature controls within the airplane. The ventilation system includes individual air outlets 410 direct to the seating areas and cabin air outlets 420 for overall cabin air circulation. One or more HOCL mister 430 disperses the non-toxic HOCL COVID-19 disinfectant with HOCL misting into airplane ventilation system 440 ducts. The airplane ventilation system blowers force the dispersed HOCL mist throughout the cabin as shown in the airplane ventilation air flow circulation 450 of one embodiment.

### A Public Restroom HOCL Disinfectant Mist:

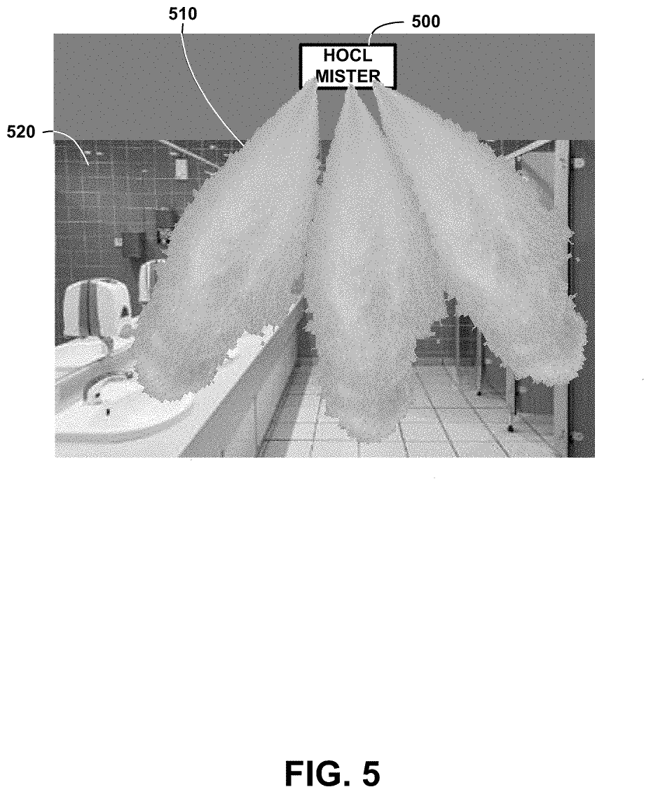

FIG. 5 shows for illustrative purposes only an example of a public restroom HOCL disinfectant mist of one embodiment. FIG. 5 shows a public restroom 520 that not only requires hygienic cleaning but at least during the pandemic COVID-19 disinfection of tiled surfaces, porcelain fixtures, fixture and stall handles and the air within the restroom. FIG. 5 shows a HOCL mister 500 positioned in the public restroom 520 for providing a HOCL disinfectant mist 510 periodically during building operating hours and can be scheduled during normal cleaning operations. The restroom attendant can initiate an aerosol mist application when activating the HOCL misting system using the HOCL misting system control app when safe 230 of FIG. 2 of one embodiment.

### A Movie Theater HOCL Disinfectant Mist:

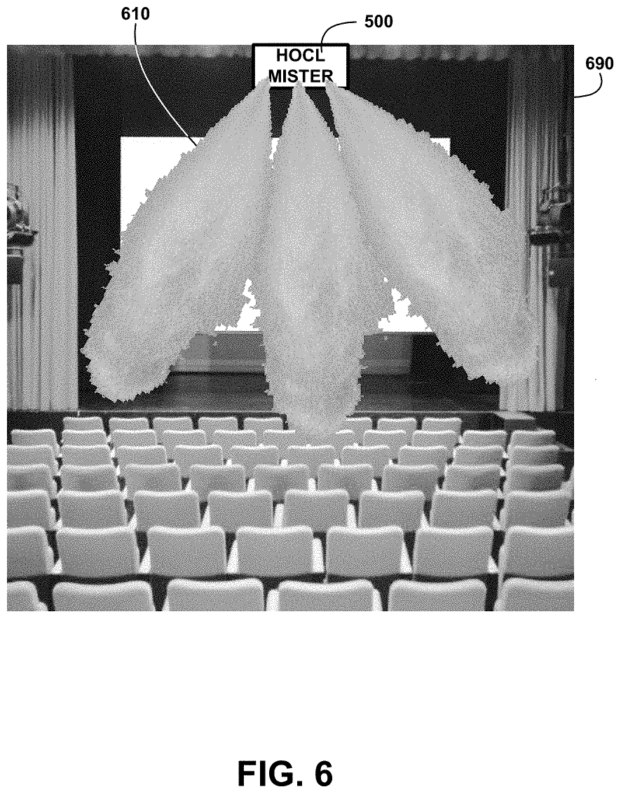

FIG. 6 shows for illustrative purposes only an example of a movie theater HOCL disinfectant mist of one embodiment. FIG. 6 shows a movie theater 690 without movie goers before the theater opens, after it closes and in between screening times. At least one HOCL mister 500 is positioned in the movie theater 690 to reach full disinfection coverage of the seating and aisles. An application of a HOCL disinfectant mist 610 will not only disinfect the seating, arm rest and aisles of foot traffic but also the air within the theater. In another embodiment the at least one HOCL mister 500 can be positioned to disperse within the intake ducting of the theater ventilation system of one embodiment.

### Misting Diffused HOCL Droplets:

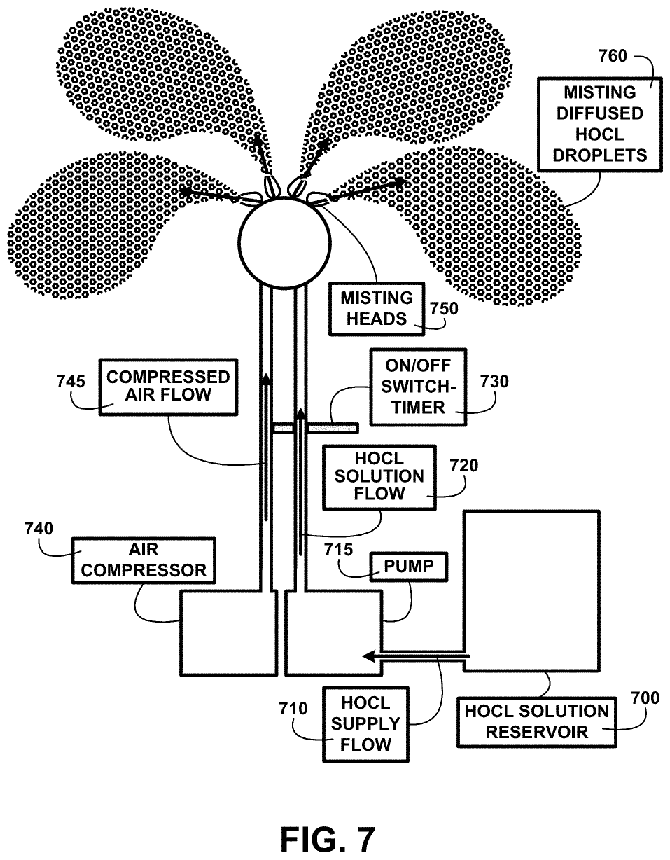

FIG. 7 shows for illustrative purposes only an example of misting diffused HOCL droplets of one embodiment. FIG. 7 shows a COVID-19 disinfectant misting system including a HOCL solution reservoir 700 for providing a HOCL supply flow 710 to a pump 715. The pump 715 provides a pressurized HOCL solution flow 720 when an on/off switch-timer 730 is in the on position to misting heads 750. An air compressor 740 provides a compressed air flow 745 to further pressurize to propel the pressurized HOCL solution flow 720 through the misting heads 750 to apply within a large commercial area misting/aerosolizing disinfectant in the form of misting diffused HOCL droplets 760. The misting diffused HOCL droplets will form a mist particle size 50 microns or less, ideally, 7 to 11 microns. The air compressor 740 provides a compressed air flow 745 at 1 to 2 CFM. The misting system air pressure will range from 20 psi to 40 psi for the flow rate of one embodiment.

HOCL Contact with a SARS-CoV2 Virus Cell Lipid Layer:

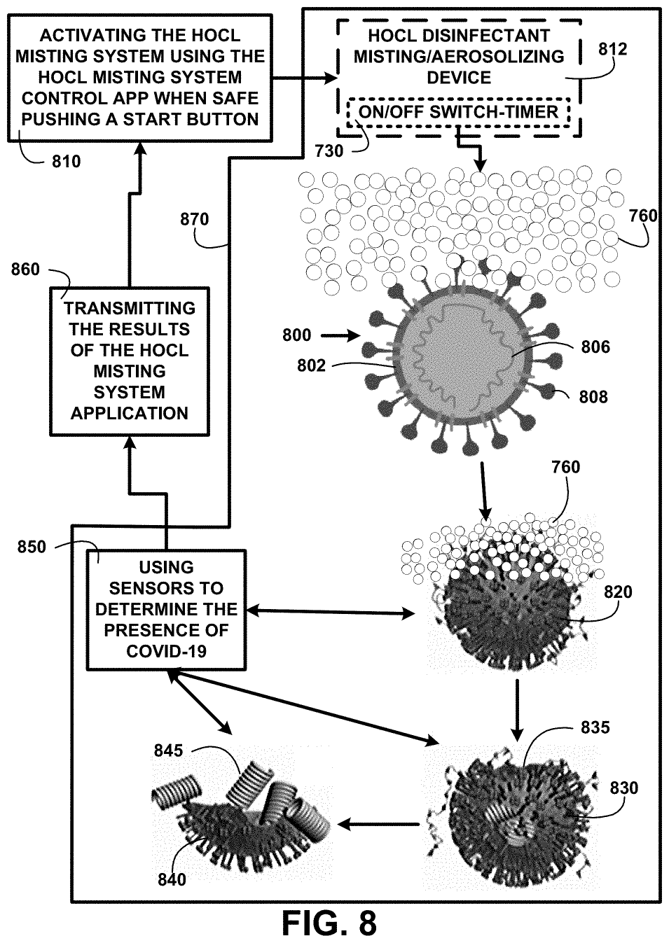

FIG. 8 shows for illustrative purposes only an example of HOCL contact with a SARS-CoV2 virus cell lipid layer of one embodiment. FIG. 8 shows large commercial area venue 870 with an installation of a HOCL disinfectant misting/aerosolizing device 812 including the on/off switch-timer 730. The user can start activating the HOCL misting system using the HOCL misting system control app when safe pushing a start button 810 for destroying SARS-CoV2 virus cells 800. The on/off switch-timer 730 will continue the misting operation for a predetermined period of time to provide full coverage of the large commercial area venue 870.

The SARS-CoV2 virus cell 800 includes a lipid layer 802 encasing the SARS-CoV2 virus RNA 806. Spaced around the lipid layer 802 are S-protein 808 structures. FIG. 8 also shows the misting diffused HOCL droplets 760 starting to contact the SARS-CoV2 virus cell 800. HOCL droplets contact with SARS-CoV2 virus cell lipid layer 820 begins to attack the lipid layer 802.

HOCL will breach the lipid layer 802 leaving SARS-CoV2 virus cell RNA exposed 830. Showing is the lipid layer destroyed by HOCL 835. The result is the SARS-CoV2 virus cell destroyed by HOCL 840 when SARS-CoV2 virus cell RNA is deactivated 845. This destroying of the SARS-CoV2 virus cell takes place on SARS-CoV2 virus cells on a surface and when the HOCL droplets contact with SARS-CoV2 virus cell suspended in the air. The result is swift and prevents air borne SARS-CoV2 virus cells from re-infecting surfaces after a surface only disinfection has been done. The HOCL misting system includes using sensors to determine the presence of COVID-19 850 before, during and after the HOCL misting application. The sensors are configured for transmitting the results of the HOCL misting system application 860 to the HOCL misting system control app to determine if a reapplication will be performed to destroy any remaining SARS-CoV2 virus cells of one embodiment.

### A Hand-Held/Portable Misting Sprayer:

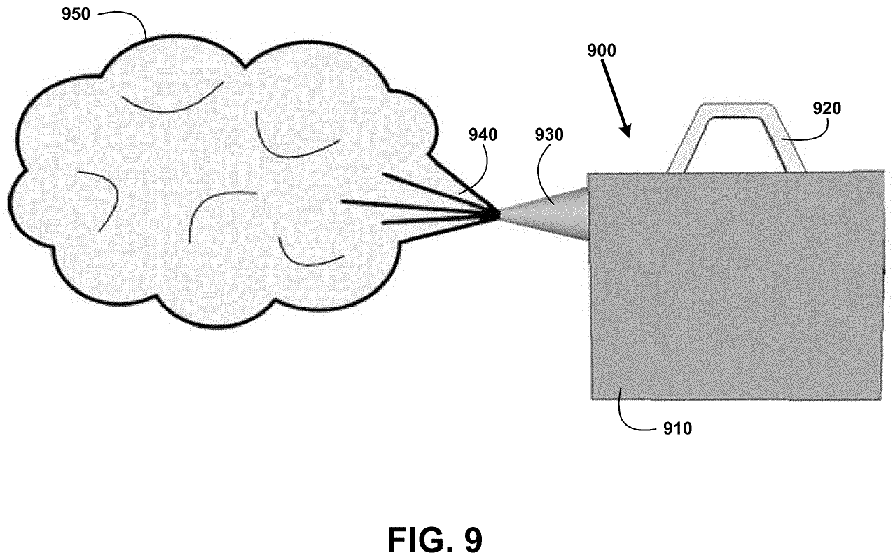

FIG. 9 shows for illustrative purposes only an example of a hand-held/portable misting sprayer of one embodiment. FIG. 9 shows a hand-held/portable misting sprayer 900. The hand-held/portable misting sprayer 900 includes a sprayer portable case 910, sprayer portable case handle 920, and spray nozzle 930 for emitting a pressurized spray 940 to create a pressurized spray diffused mist 950.

The hand-held/portable misting sprayer 900 is operated for applying non-toxic HOCL to surfaces of objects for destroying airborne and surfaces contacted with the COVID-19 SARS-CoV-2 virus and other viruses. An electrolyzed water system is used to generate hypochlorous acid from just table salt, water and electricity. No personal protective gear is required. Electrolysis is a technique that uses a direct electric current (DC) to drive an otherwise non-spontaneous chemical reaction. A pH of the solution weakly acidic to neutral produces a free chlorine solution dominated by hypochlorous acid. Hypochlorous acid is effective with a pH level between pH 3 and pH 7.5.

Hypochlorous acid does not cause irritation to eyes and skin. Even ingested it causes no harm. Because it is so safe, it is the ideal sanitizer for direct food sanitation and food contact surfaces. It is also ideal in healthcare where it is used for wound cleansing, eye drops, and patient room disinfection. HOCL is non-toxic and non-hazardous. HOCI is made from salt and once deactivated, it turns back into saline making it environmental safe. HOCI is safe on most fabrics.

The non-toxic and non-hazardous nature of HOCL allows it to be used on application to humans and other mammals for example pets. The non-toxic HOCL mist destroys airborne and surface COVID-19 and other viruses on contact. The hand-held/portable misting sprayer 900 can be used for example for spraying passengers boarding an airplane or a cruise ship. The sprayed HOCL posed no threat to the person or their clothes.

Airborne microbial pathogens can settle on pet fur, a person's clothing, any surface exposed to the air. The surfaces contaminated with SARS-CoV-2 virus and other viruses pose a threat of spreading the virus to others by contact with that contaminated surface. Periodic spraying of pets will not harm the pet and disinfect their fur to prevent spreading any virus to people petting the animal. Athletes before playing contact sports can be sprayed to reduce spreading Covid-19 to other players. Restaurants can spray not only the food cooking and preparation equipment but also the food as the HOCL sprayed using the hand-held/portable misting sprayer 900 is non-toxic and non-hazardous. The HOCL non-toxic and non-hazardous is safe for also spraying tables and chairs, utensils, drinking glasses and other items that both staff and customer would be coming in contact with during their work or visit.

Coupled to the hand-held/portable misting sprayer 900 is at least one portable rechargeable battery pack for supplying power to operate elements of the hand-held portable misting sprayer. An HOCL reservoir is coupled to the hand-held portable misting sprayer for containing a volume of HOCL for suppling spraying operations. A pressuring motor is coupled to the hand-held portable misting sprayer for pressurizing HOCL through a spray nozzle for creating a diffused mist.

Electronic processing and communication modules are coupled to the hand-held portable misting sprayer and components for gathering, recording, processing data gathered, and transmitting the data to external digital devices wirelessly. A hand-held/portable misting sprayer app is provided for use on user digital devices for receiving and processing data transmitted from the hand-held portable misting sprayer of one embodiment.

### Hand-Held/Portable Misting Sprayer System:

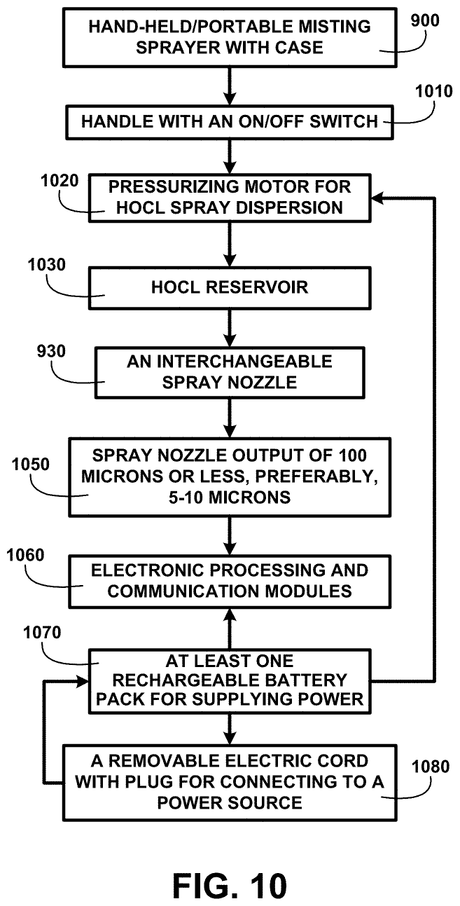

FIG. 10 shows a block diagram of an overview of a hand-held/portable misting sprayer system of one embodiment. FIG. 10 shows the hand-held/portable misting sprayer with case 900 including a handle with an on/off switch 1010. The handle with an on/off switch 1010 is conveniently located for a user to be able to turn on or off the hand-held/portable misting sprayer with their thumb of the hand holding the sprayer. A pressurizing motor for HOCL spray dispersion 1020 draws HOCL solution from a HOCL reservoir 1030. The pressurizing motor conveys HOCL through an interchangeable spray nozzle 930 to create a spray nozzle output of 100 microns or less, preferably, 5-10 microns 1050. The pressurized HOCL at a predetermined pressure forms a diffused mist after passing through the spray nozzle that passes through the air and deposits a fine coating on surfaces.

The non-toxic HOCL mist destroys airborne and surface COVID-19 and other viruses on contact. Applying the HOCL using the hand-held/portable misting sprayer provides a user an apparatus for quickly disinfecting large areas quickly as the user moves through the area. The electronic processing and communication modules 1060 gathered data provides details of the HOCL application that is useful in establishing a disinfection regimen.

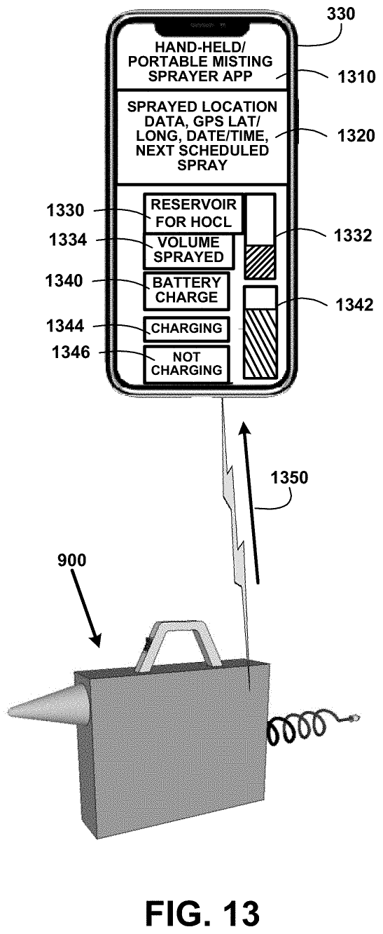

Sensors 1062 are coupled to the HOCL reservoir 1030 for analyzing and measuring pH levels and PPM (Parts per Million) of the HOCL. The sensors 1062 are electronically coupled to the electronic processing and communication modules 1060 to automatically transmit data on pH levels and PPM of the HOCL to the electronic processing and communication modules 1060. The electronic processing and communication modules 1060 automatically transmit the pH levels and PPM of the HOCL to the hand-held/portable misting sprayer app 1310 of FIG. 13 on a user digital device.

The hand-held/portable misting sprayer app 1310 of FIG. 13 displays the current pH levels and PPM of the HOCL to inform the user of the automated adjusts to the HOCL solution to predetermined pH level and PPM concentrations. An acid supply tank 1063, a base chemical supply tank 1064 and a concentrated HOCL supply tank 1065 are electronically activated by the sensors 1062 to deposit into the HOCL reservoir 1030 in the quantities determined by the sensors 1062 analysis to adjust the solution to the predetermined pH level and PPM concentration of the HOCL solution.

The hand-held/portable misting sprayer app 1310 of FIG. 13 displays the automatically completed adjusted pH level and PPM concentration conditions and a notice to the user that the HOCL solution is ready for applying using the hand-held/portable misting sprayer 900.

At least one rechargeable battery pack for supplying power 1070 to the pressurizing motor and electronic processing and communication modules 1060 is recharged periodically using a removable electric cord with plug for connecting to a power source 1080 of one embodiment.

### A Hand-Held/Portable Misting Sprayer Exterior View:

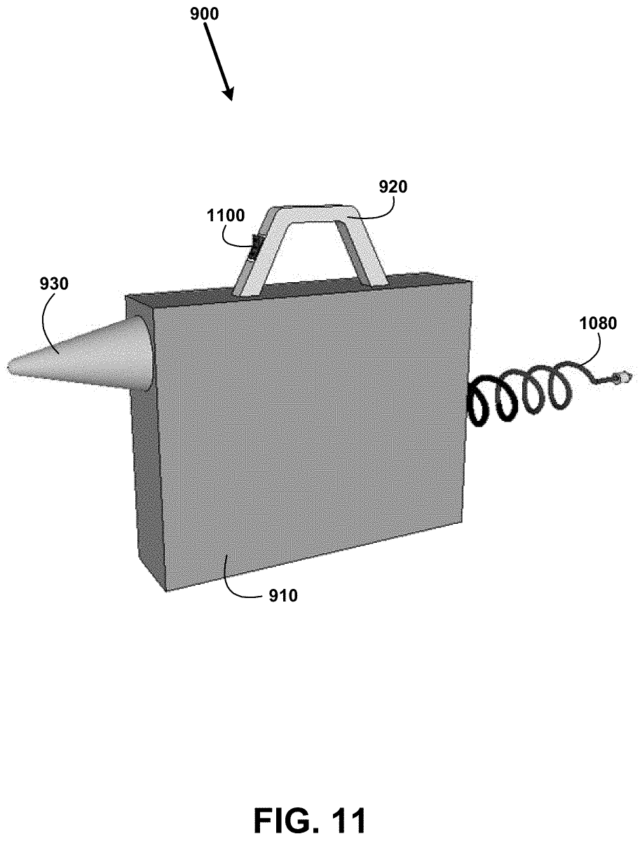

FIG. 11 shows for illustrative purposes only an example of a hand-held/portable misting sprayer exterior view of one embodiment. FIG. 11 shows the hand-held/portable misting sprayer 900, sprayer portable case 910, sprayer portable case handle 920, spray nozzle 930, handle on/off switch 1100 and electric cord for power source 1080. This external view shows the compact portable characteristics that are easily carried in a hand and moved about to disperse the HOCL mist in a large area quickly of one embodiment.

### A Hand-Held/Portable Misting Sprayer Interior View:

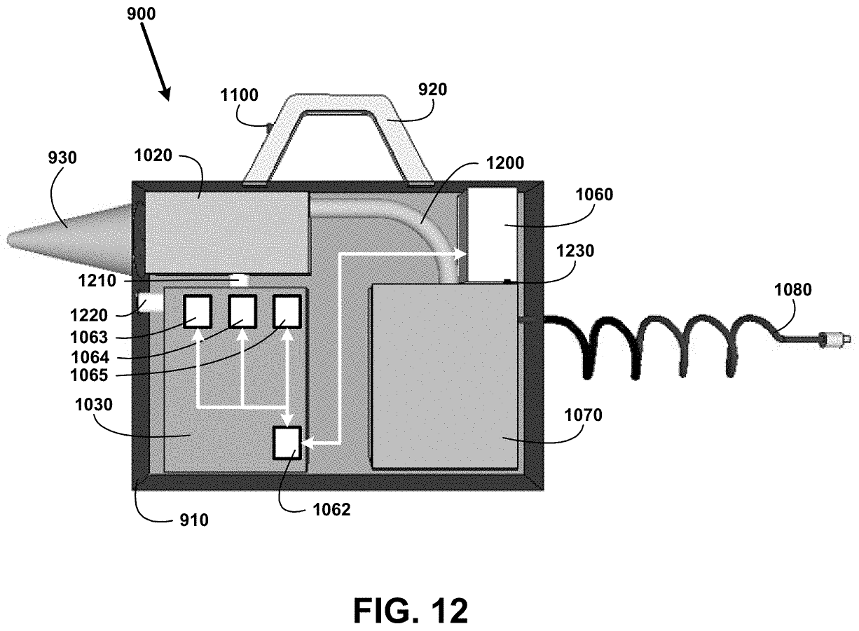

FIG. 12 shows for illustrative purposes only an example of a hand-held/portable misting sprayer interior view of one embodiment. FIG. 12 shows the hand-held/portable misting sprayer 900 with one side of the sprayer portable case 910 removed for illustration purposes. The sprayer portable case handle 920 is seen with the conveniently located handle on/off switch 1100. The spray nozzle 930 at one end is shown relative to the pressurizing motor for HOCL spray dispersion 1020. The reservoir for HOCL 1030 is filled through a reservoir for HOCL fill tube 1220 that has a removable stopper that opens to the outside.

A reservoir for HOCL feed tube to sprayer motor 1210 permits the motor to draw the HOCL solution from the reservoir for HOCL 1030. The at least one rechargeable battery pack for supplying power 1070 conducts electrical power through a power circuit to the spray motor 1200 and conducts electrical power through a power circuit 1230 to electronic processing and communication modules 1060. The removable electric cord with plug for connection to a power source 1080 includes at one end a connection for plugging into a recharging circuit and at the opposite end an outlet plug for connecting to an external power source of one embodiment.

A Hand-Held/Portable Misting Sprayer App:

FIG. 13 shows for illustrative purposes only an example of a hand-held/portable misting sprayer app of one embodiment. FIG. 13 shows the hand-held/portable misting sprayer 900 electronic processing and communication modules transmitting data to user digital device 1350. The user digital device 1300 has installed a hand-held/portable misting sprayer app 1310. The hand-held/portable misting sprayer 900 transmitted data includes sprayed location data, GPS lat/long, date/time, next scheduled spray 1320. The GPS lat/long means the latitude and longitude GPS coordinates. This data is displayed on the user digital device 330.

Additionally is displayed a readout for the reservoir for HOCL 1330 showing a HOCL volume indicator 1332 and a volume sprayed 1334. The volume sprayed 1334 is calculated by a digital processor of the electronic processing and communication modules 1060 of FIG. 10. The aggregated elapsed time of the on condition of the sprayer and the volume of sprayed solution passing through the spray nozzle at the predetermined pressure for a given time period is multiplied to produce the volume of solution that has been applied in the area of the GPS coordinates. The non-toxic HOCL mist destroys airborne and surface COVID-19 and other viruses on contact.

A battery charge 1340 level is displayed with a battery charge indicator 1342. This provides the user with data on when to recharge the battery pack. When the removable electric cord with plug for connection to a power source 1080 of FIG. 10 has been connected the display shows the battery pack is charging 1344 or not charging 1346 of one embodiment.

### A Hand-Held/Portable Misting Sprayer Network Platform:

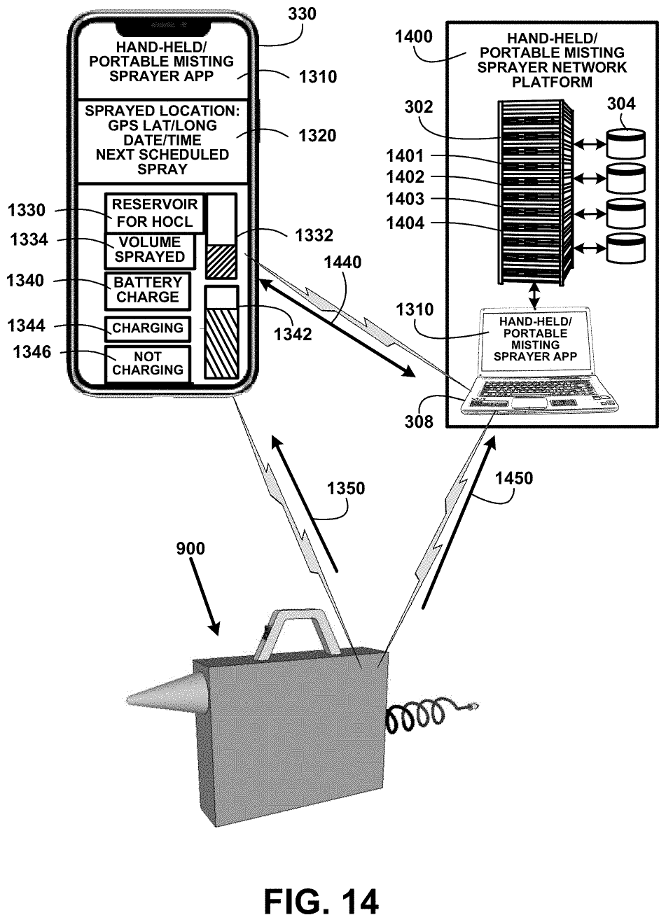

FIG. 14 shows for illustrative purposes only an example of a hand-held/portable misting sprayer network platform of one embodiment. FIG. 14 shows the hand-held/portable misting sprayer 900 electronic processing and communication modules transmitting data to user digital device 1350, where the user digital device 330 has the hand-held/portable misting sprayer app 1310 installed. The transmitted data includes the sprayed location data, GPS lat/long, date/time, next scheduled spray 1320, reservoir for HOCL 1330 level, HOCL volume indicator 1332, volume sprayed 1334, battery charge 1340, battery charge indicator 1342 and charging 1344 or not charging 1346 status.

The hand-held/portable misting sprayer network platform 1400 includes WI-FI communication devices 1401, Bluetooth communication devices 1402, GPS location devices 1403, and digital street maps with GPS grid coordinates 1404. The WI-FI communication devices 1401 will provide continual Covid-19 infection hot spot monitoring. The hot spot locations will be updated and recorded in the plurality of databases 304.

For example first responders will receive hot spot alerts transmitted from the hand-held/portable misting sprayer network platform 1400. The hot spot alerts will be received on a first responder's digital device 330 on the hand-held/portable misting sprayer app 1310. The hand-held/portable misting sprayer app 1310 will display digital street maps with GPS grid coordinates 1404 showing the hot spot GPS location using the GPS location devices 1403.

This will aid first responders to know where the hand-held/portable misting sprayer 900 applications of the HOCL disinfectant will assist in reducing the spread of Covid-19 and other airborne and surface viruses. The non-toxic HOCL mist destroys airborne and surface COVID-19 and other viruses on contact.

The hand-held/portable misting sprayer 900 with the hand-held/portable misting sprayer app 1310 will wirelessly transmit 1450 data to a network computer 1440. The data is processed in a digital server 302, recorded in a plurality of databases 304 and processed to create a disinfection record for each sprayed location of one embodiment.

### Electronic Processing and Communication Modules:

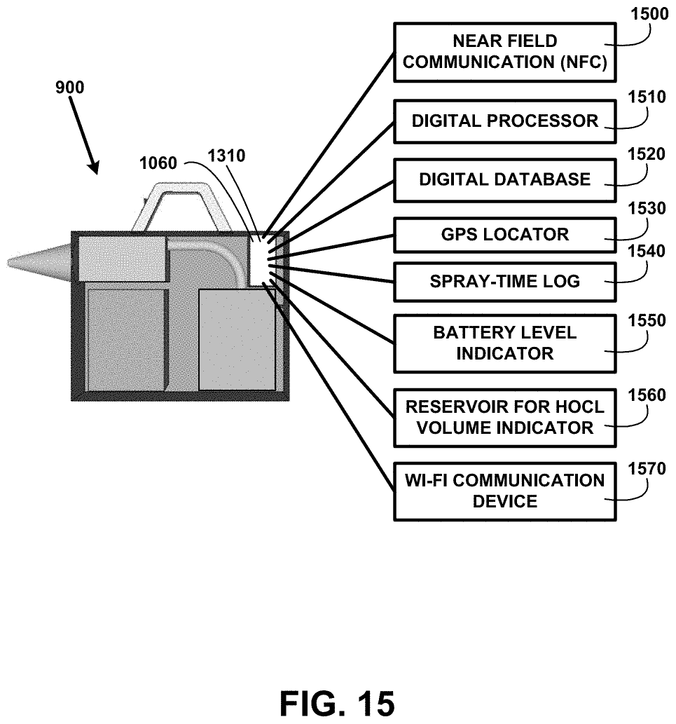

FIG. 15 shows for illustrative purposes only an example of electronic processing and communication modules of one embodiment. FIG. 15 shows the hand-held/portable misting sprayer 900 electronic processing and communication modules 1060 that include a near field communication (NFC) 1500 device, digital processor 1510, digital database 1520, GPS locator 1530, spray-time log 1540 processor, battery level indicator 1550 device, reservoir for HOCL volume indicator 1560 device and WI-FI communication device 1570.

The electronic processing and communication modules 1060 provide real-time and historical data of the disinfection regimen being implemented for a large area facility. During times of a pandemic the disinfection regimen and data supporting the actual application of the regimen may provide information that will allow a facility to maintain operation. Outside of a pandemic situation the application of the disinfection regimen will reduce employee and customer potential illness.

Person to person spreading of a virus and other microbial organisms can be via contamination of surfaces and air by respiratory and tactile means. The non-toxic HOCL mist destroys airborne and surface COVID-19 and other viruses on contact. The hand-held/portable misting sprayer 900 provides a quick, cost effective method of decontamination that can reduce employee sick days and prevent the spread of those that may have contracted the illness elsewhere from spreading to others of one embodiment.

### Sprayer Application on a Ship's Bridge:

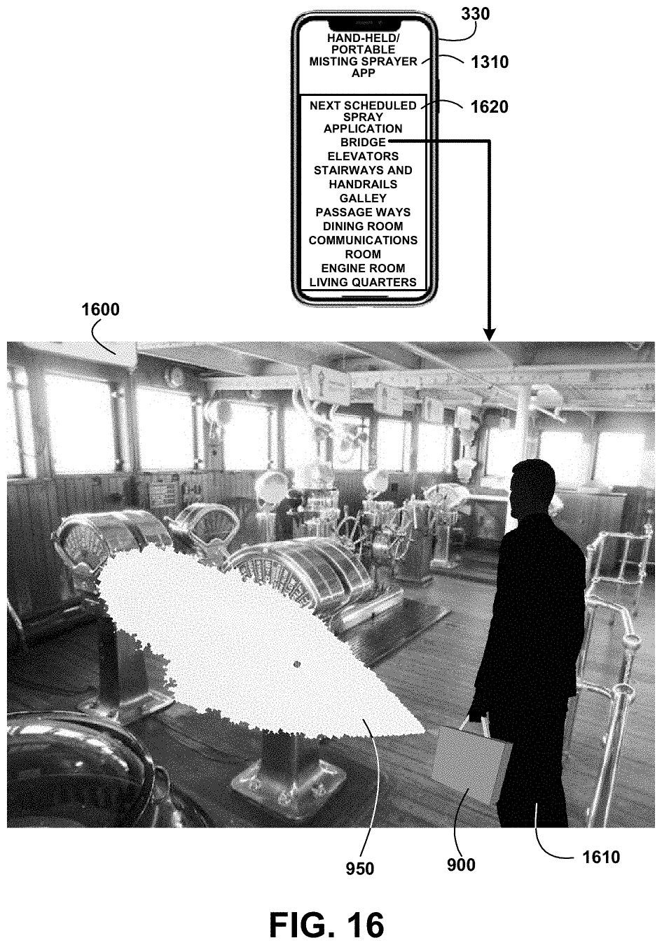

FIG. 16 shows for illustrative purposes only an example of a hand-held/portable misting sprayer application on a ship's bridge of one embodiment. FIG. 16 shows an example of a large commercial area that is composed of many compartmentalized sections on many levels making the installation of the fixed decontamination system impractical. A ship whether a cargo vessel, a cruise ship or a military ship contain many passage ways on a number of decks and compartments for ship operations, living quarters, dining, food preparation and more. FIG. 16 shows for example a ship's bridge 1600. A modern ship's bridge 1600 contains touch screen displays, numerous control panels and other electronic equipment that crew member are touching regularly.

Applying a liquid disinfectant from a spray bottle runs the risk of the liquid seeping into the electrical systems and shorting out the equipment. Hand wiping the same equip can inadvertently lead to a button or touch screen click section being activated that should not be indiscriminately activated. A crew member 1610 using the hand-held/portable misting sprayer 900 avoids those situations when applying the pressurized spray diffused mist 950.

The user digital device 330 with the hand-held/portable misting sprayer app 1310 will display the next scheduled spray application 1620 to guide the crew member 1610 on the disinfecting rounds listing the areas for application for example in this instance the bridge, elevators, stairways and handrails, galley, passage ways, dining room, communications room, and engine room. The non-toxic HOCL mist destroys airborne and surface COVID-19 and other viruses on contact. The hand-held/portable misting sprayer 900 application of HOCL can be used for other large area facilities including for example hospitals, hotels, office buildings, school class rooms, factories, libraries and many more of one embodiment,

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1-15. (canceled)

16. An apparatus, comprising:
a hand-held/portable misting sprayer device configured for producing a projected mist of a HOCL solution;
wherein the hand-held/portable misting sprayer device is coupled to a pressurizing motor;
wherein the pressuring motor is coupled to a reservoir of the HOCL solution;
wherein the pressurizing motor is powered by an electrical coupling to at least one rechargeable battery pack;
an interchangeable spray nozzle coupled to the pressurizing motor and configured for diffusing the projected mist of a HOCL solution; and
a plurality of electronic processing and communication modules coupled to the hand-held/portable misting sprayer device and configured for gathering, recording and transmitting data on an application of the diffused projected mist in real-time.

17. The apparatus of claim 16, further comprising the interchangeable spray nozzle is configured for producing a spray nozzle output of 100 microns or less, preferably, 5-10 microns.

18. The apparatus of claim 16, further comprising a hand-held/portable misting sprayer app configured for receiving and displaying the plurality of electronic processing and communication modules gathered, recorded and transmitted data on an application of the diffused projected mist in real-time on a user digital device.

19. The apparatus of claim 16, further comprising the pressurizing motor is configured for passing a volume of the HOCL solution through the spray nozzle at the predetermined pressure.

20. The apparatus of claim 16, further comprising a hand-held/portable misting sprayer app configured for receiving and displaying the plurality of electronic processing and communication modules gathered, recorded and transmitted data on an application of the diffused projected mist in real-time on a network computer coupled to a hand-held/portable misting sprayer network platform.
